(19) United States
(12) Patent Application Publication
(10) Pub. No.: US 2013/0064710 A1
(43) Pub. Date: Mar. 14, 2013

(54) PLASMA APPARATUS FOR BIOLOGICAL DECONTAMINATION AND STERILIZATION AND METHOD FOR USE

(71) Applicant: Jamey D. JACOB, Stillwater, OK (US)

(72) Inventor: Jamey D. JACOB, Stillwater, OK (US)

(21) Appl. No.: 13/411,311

(22) Filed: Mar. 2, 2012

Related U.S. Application Data
(60) Provisional application No. 61/449,321, filed on Mar. 4, 2011.

(51) Int. Cl. H05H 1/48; A61L 9/22

(52) U.S. Cl. 422/4; 422/186.21

(57) ABSTRACT

A device having dielectric layer with opposite sides and a length. First and second electrodes are each on an opposite side of the dielectric layer and offset along the length of the dielectric layer. A voltage source selectively provides a first voltage on the first electrode and a second voltage on the second electrode such that plasma is generated along the dielectric layer, the plasma providing a decontamination mechanism of adjacent air, and movement of the adjacent air along the dielectric layer.

### CROSS REFERENCE TO RELATED APPLICATIONS

This application claims the priority of U.S. Provisional Patent Application No. 61/449,321 entitled “PLASMA APPARATUS FOR BIOLOGICAL DECONTAMINATION AND STERILIZATION,” filed Mar. 4, 2011, the contents of which are hereby incorporated by reference.

### FIELD OF THE INVENTION

This disclosure is related to plasma technologies in general and, more particularly, to plasma apparatus for biological decontamination and/or sterilization.

### BACKGROUND OF THE INVENTION

Plasma actuators are zero-net mass flux (ZNMF) devices that use atmospheric pressure electrical discharges. These discharges are from a class that includes corona discharges, dielectric barrier discharges (DBDs), glow discharges and arc discharges. Plasma is further known to be a sterilization medium for a number of biological agents through some combination of the mechanisms of heat, ultraviolet radiation, ionization, etc. However, the items to be sterilized must be placed within the plasma itself, possibly damaging the device to be sterilized and limiting the scope and efficacy of the sterilization volume.

What is needed is a system and method for addressing the above, and related, concerns.

### SUMMARY OF THE INVENTION

The invention of the present disclosure, in one aspect thereof, comprises a device having a dielectric layer with opposite sides and a length. First and second electrodes are each on an opposite side of the dielectric layer and offset along the length of the dielectric layer. A time-varying high voltage source selectively provides a first voltage on the first electrode and a second voltage on the second electrode such that plasma from the ambient air is generated along the dielectric layer, the plasma providing a decontamination mechanism of adjacent air, and movement of the adjacent air along the dielectric layer inherently due to the generation of the plasma.

In some embodiments the device also comprises second and third electrodes on opposite sides of the dielectric layer and offset along the length of the dielectric layer. The voltage source selectively provides a third voltage on the third electrode and a fourth voltage on the fourth electrode such that plasma is generated along the dielectric layer, the plasma providing a decontamination mechanism of adjacent air, and movement of the adjacent air along the dielectric layer.

The first, second, third, and fourth electrodes and the power supply may be configured to produce a swirling effect of gasses adjacent to the dielectric layer. The dielectric layer may form a portion of a decontamination chamber, with the plasma being produced by the electrodes inside the chamber. The device may comprise means for providing a supply of contaminated air into the decontamination chamber, and means for evacuating plasma-decontaminated air from the decontamination chamber.

The invention of the present disclosure, in another aspect thereof, comprises a method including placing a first electrode on a first side of a dielectric material, placing a second electrode on a second side of the dielectric material offset from the first electrode, exposing the first side of the dielectric material to a contaminated gas, and applying a sufficient voltage differential to the first and second electrodes as to produce a plasma stream on the first side of the dielectric material to decontaminate the gas.

The method may also include providing a decontamination chamber with the first electrode exposed to an interior thereof, introducing the contaminated gas into the decontamination chamber, and evacuating decontaminated gas from the decontamination chamber after exposure to plasma.

In some embodiments the method may include placing a third electrode on the first side of a dielectric material, placing a fourth electrode on the second side of the dielectric material offset from the third electrode, applying a sufficient voltage differential to the third and fourth electrodes as to produce a second plasma stream on the first side of the dielectric material to decontaminate the gas, and arranging the first, second, third, and fourth electrodes such that the first and second plasma steams are directed in opposite directions to as to produce a swirling effect of the gas near the dielectric layer.

In another embodiment there is provided a dielectric layer of arbitrary size, a first and a second electrode each on an opposite side of the dielectric layer, with one of the electrodes having at least one free edge, and a single voltage source that selectively provides a first voltage on the first electrode and a second voltage on the second electrode such that plasma is generated along the dielectric layer, the plasma providing a decontamination mechanism of adjacent air, and movement of the adjacent air along the dielectric layer.

In still another embodiment there is provided a dielectric layer substantially as described above which is non-planar.

In still another embodiment there is provided a dielectric layer of arbitrary size and shape that may be planar or not, a first and a second electrode each positioned on an opposite side of the dielectric layer, with one of the electrodes having at least one free edge, and a single voltage source that selectively provides a first voltage on the first electrode and a second voltage on the second electrode such that plasma is generated along the dielectric layer proximate an edge of one of the electrodes, the plasma providing a decontamination mechanism of adjacent air, and movement of the adjacent air along the dielectric layer.

The foregoing has outlined in broad terms the more important features of the invention disclosed herein so that the detailed description that follows may be more clearly understood, and so that the contribution of the instant inventors to the art may be better appreciated. The instant invention is not limited in its application to the details of the construction and to the arrangements of the components set forth in the following description or illustrated in the drawings. Rather the invention is capable of other embodiments and of being practiced and carried out in various other ways not specifically enumerated herein. Additionally, the disclosure that follows is intended to apply to all alternatives, modifications and equivalents as may be included within the spirit and the scope of the invention as defined by the appended claims. Further, it should be understood that the phraseology and terminology employed herein are for the purpose of description and should not be regarded as limiting, unless the specification specifically so limits the invention.

### BRIEF DESCRIPTION OF THE DRAWINGS

FIG. 1 is a schematic diagram of one embodiment of a plasma generating device according to the present disclosure.

FIG. 2 is a schematic diagram of another plasma generating device according to the present disclosure.

FIG. 3 is a schematic diagram of a plasma decontamination system according to the present disclosure.

FIG. 4 contains some example relative positions of upper and lower conductors that would be suitable for use with the instant invention.

FIG. 5 contains schematic illustrations of linear and annular examples of the instant invention.

FIG. 6 contains additional details of an annual embodiment.

FIG. 7 illustrates relative motive force for some different configurations of the embodiment of FIG. 6.

FIG. 8 contains schematic illustrations of asymmetrical motive force that will typically be produced by the embodiment of FIG. 6.

FIG. 9 contains still another embodiment of the instant invention wherein multiple annular electrodes are used.

FIG. 10 illustrates a cross sectional view of another annular embodiment of the instant invention.

FIG. 11 contains schematic illustrations of some other configurations of the instant invention.

### DETAILED DESCRIPTION OF THE PREFERRED EMBODIMENTS

In various embodiments of the present disclosure a plasma actuator is used for biological decontamination. Some embodiments of the present disclosure are based on the one atmosphere uniform glow discharge or single dielectric barrier discharge concept of cold plasma generation.

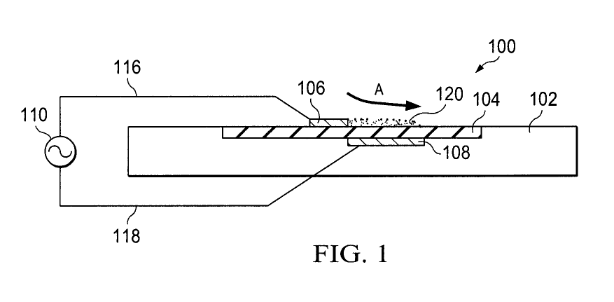

Referring now to FIG. 1, a schematic diagram of one embodiment of a plasma generating device according to the present disclosure is shown. In the embodiment of FIG. 1, the device 100 includes a substrate 102 onto which the various other components described herein may be attached. As will be explained in greater detail below, the substrate 102 could be a portion of a chamber or enclosure. A suitable substrate 102 would be a non-conductive, impermeable material that is resistant to high temperatures or gas species. Glass, acrylic or phenolic materials are examples of acceptable materials.

Integrated with the substrate 102, or forming a part of the substrate 102, is a dielectric layer 104. The dielectric layer 104 could be formed, by way of example only, from any material with a low dielectric constant such as PTFE or kapton.

An electrode 106 is situated along a top surface of the dielectric layer 104. A second electrode 108 is situated along a lower surface of the dielectric layer 104. It can be seen that the electrodes 106, 108, are at least somewhat offset from one another along a length of the dielectric layer 104. The electrodes 106 and 108 might be made of copper or any other material with suitable conductivity.

The electrode 106 attaches to a voltage source 110 by an electrical lead 116. The electrode 108 attaches to the voltage source 110 by an electrical lead 118. In the present embodiment, the voltage source 110 may include a power supply as well as any necessary transformers or circuit conditioning components to enable generation of plasma by application of sufficient voltage between the leads 106, 108 on the surface of the dielectric layer 104. In the present embodiment, a plasma region 120 develops between the first electrode 106 and the second electrode 108. The plasma region 120 also provides a motive force for any adjacent gases in the direction of the arrow “A”.

Various duty cycles and voltages may be utilized to generate plasma. In the present embodiment, various voltages, frequencies and duty cycles have been tested and found to be operational. By way of example only, these include voltages in the range of 5 to 50 kV at frequencies of 1,000 to 10,000 Hz at a 10% to 100% duty cycle at modulated frequencies of 1, 2, 5, 10, 100, 500 and 5000 Hz. It will be appreciated that various flow rates and associated decontamination characteristics can be generated by adjusting the duty cycle voltage and frequency of the applied voltage. In application, the limit is most likely to be the durability of the materials used to construct the device 100 and the available power supply. For example, if operating from commercial power, higher voltages may be available than if operating from battery power.

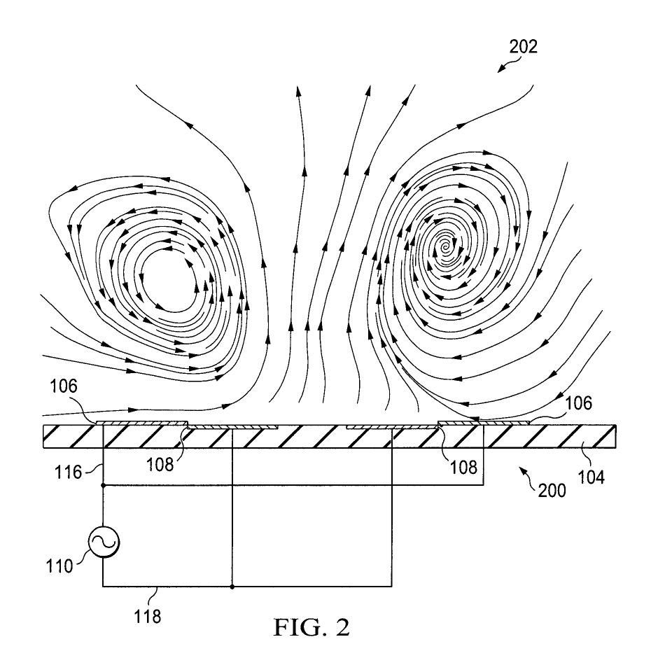

Referring now to FIG. 2, a schematic diagram of another plasma generating device according to the present disclosure is shown. The device 200 is similar in construction and operation to the device 100 of FIG. 1. In the present device, two upper electrodes 106 are attached opposite a dielectric layer 104, and are offset from a pair of lower electrodes 108. Electrical lead 116 attaches the upper electrodes 106 to the voltage source 110 and a lower electrical lead 118 attaches the lower electrodes 108 to the voltage source 110.

In the present embodiment, it will be appreciated that, due to the configuration of the electrodes 106 relative to the electrodes 108, flow regions that are pointed in substantially opposite directions will be achieved. Thus, each electrode pair 106, 108, will generate plasma as well as a motive force pointed inward according to FIG. 2. This will cause a swirling effect of any adjacent gases as illustrated by the exemplary flow lines 202.

In FIG. 2, both of the upper electrodes 106 are shown attached to a common voltage line 116. Similarly, the lower electrodes 108 are shown attached to a common voltage line 118. Thus, in operation, in this embodiment the upper electrodes 106 will always be at the same voltage potential while the lower electrodes 108 will likewise share a voltage potential. However, it is understood that other configurations are possible. For example, both of the upper electrodes 106 need not necessarily be operated at the same voltage level. Similarly, the lower electrodes 108 could be attached to different voltage levels. In this manner the device 200 may be operated in a pulsing fashion where the gas flow is first in one direction, and then in another. It will be appreciated that both of the aforedescribed exemplary operating methods will result in a thorough mixing of gases next to and around the device 200. Thus, over time the adjacent gases will be exposed to the plasma generated by the device and the air thereby decontaminated from biological agents.

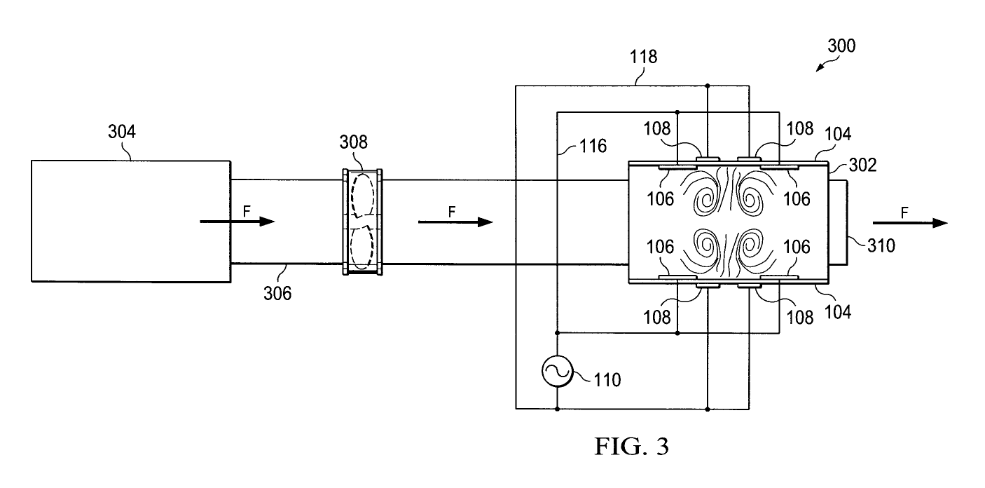

Referring now to FIG. 3, a schematic diagram of plasma decontamination system according to the present disclosure is shown. The plasma decontamination system 300 comprises a plasma decontamination chamber 302. This chamber 302 may have a plurality of inner electrodes 106 separated from a plurality of outer electrodes 108 by a dielectric layer 104. The dielectric layer 104 may be enclosed by a substrate (not shown).

The inner electrodes 106 may attach to a voltage source 110 by a lead 116. The outer electrodes 108 may attach to the voltage source 110 by a lead 118. The plasma decontamination system 300 operates in a manner similar to those previously described in that voltages will be applied to the plurality of inner electrodes 106 and outer electrodes 108 generating plasma inside the plasma decontamination chamber 302. The motive forces provided by the plasma generation will serve to mix and swirl gas within the plasma decontamination chamber 302 such that the gases inside of the chamber 302 may be substantially completely decontaminated from biological agents.

In some embodiments, the motive force for drawing contaminated air into the plasma decontamination chamber 302, and expelling decontaminated air, will be entirely due to the location and configuration of the plasma generating electrodes 106, 108 in and on the plasma decontamination chamber 302. However, in other embodiments, a separate flow control system may be utilized that provides for selective introduction of contaminated gases into the decontamination chamber 302 from a contamination source 304. The contamination source 304 could be naturally or otherwise occurring bacteria or viruses, medical waste, sewage or any number of sources which generate air containing bio-contaminants. In the present embodiment, the gases flow generally from the contamination source 304 in the direction of the arrows “F”.

A conduit 306 is provided between the plasma decontamination chamber 302 and the contamination source 304. A fan 308 may be provided that produces vacuum toward the contamination source 304, and positive pressure toward the plasma decontamination chamber 302. The fan 308 or other flow driving device may operate in an open-loop configuration or may be selectively activated such that air within the decontamination chamber 302 has sufficient time for exposure to plasma to achieve a satisfactory level of decontamination. An exit conduit 310 may be provided for moving the decontaminated gas away from the decontamination chamber 302. In some embodiments, the exit conduit 310 will merely function as a selectively closeable valve to prevent air from escaping the decontamination chamber 302 until sufficiently and effectively decontaminated.

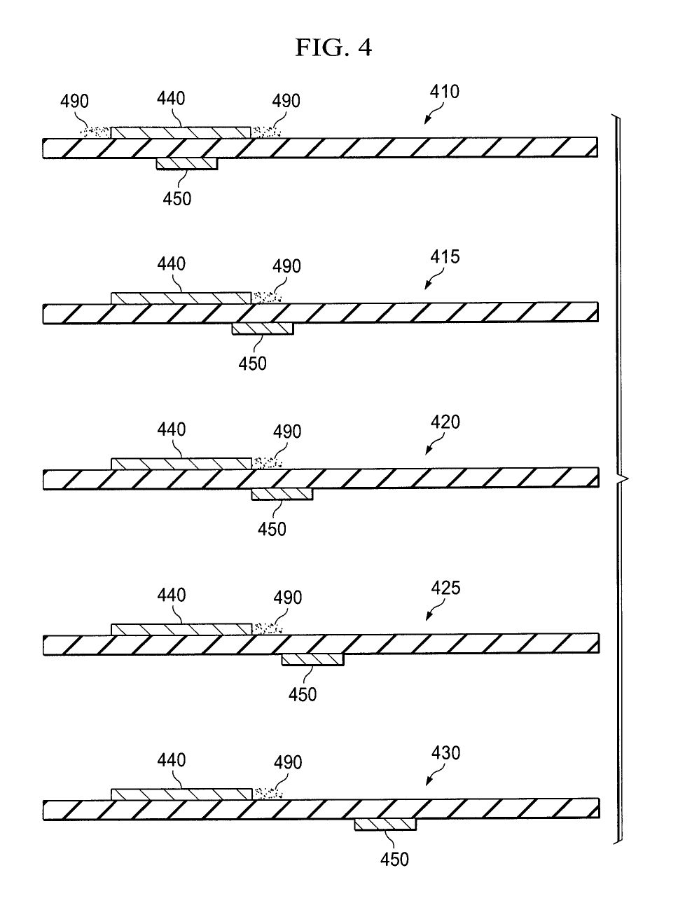

FIGS. 4 through 11 illustrate additional examples of the instant invention. In FIG. 4, configuration 410 is an embodiment that would operate to generate a plasma stream 490 on both sides of the upper conductor 440 at its periphery. However, the instant inventor has found an arrangement similar to that illustrated by configuration 415, i.e., where the upper 440 and lower 450 conductors at least partially overlap, tends to produce better results. Further, and continuing with the examples of FIG. 4, configurations such as 420 to 430 tend to show generally decreasing performance as compared with configuration 415. Obviously, if the conductors are spaced sufficiently far apart the plasma generated will be negligible or zero.

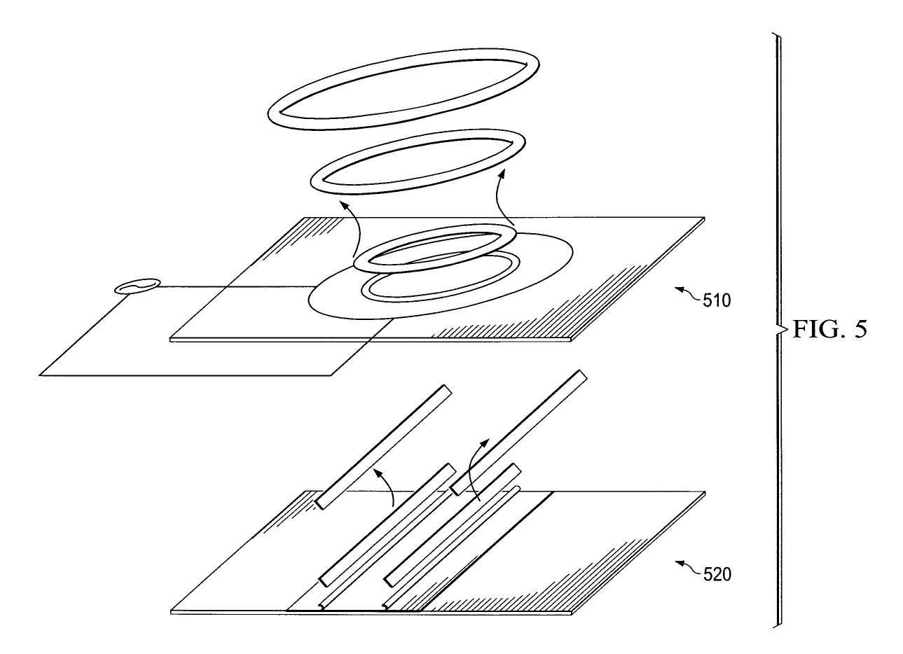

FIG. 5 contains a schematic illustration of linear 520 and annular 510 embodiments. As can be seen, in the embodiments of this figure the motive force associated with the plasma stream is in an outward (upward by reference to this figure) direction, i.e., a “blow” embodiment. That being said, if the electrical leads are reversed, a downward/inward (i.e., a “suck”) embodiment can be created.

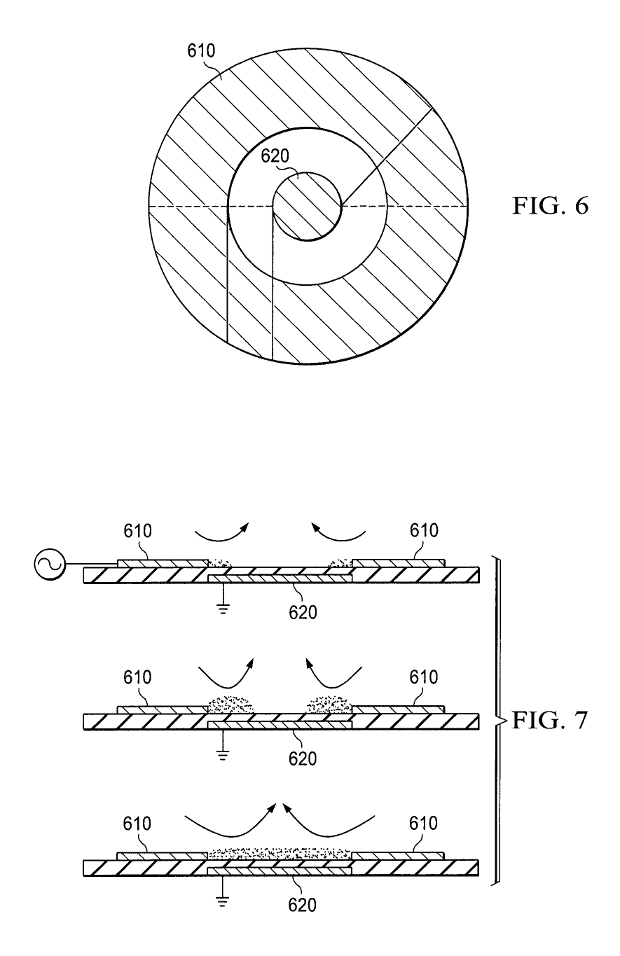

FIGS. 6 and 7 contain additional details of an annual embodiment. In the configuration of FIG. 6, note that the amount of plasma generated and the corresponding motive force can be varied by increasing the voltage differential that is supplied to the electrodes 610 and 620 as is illustrated generally in FIG. 7.

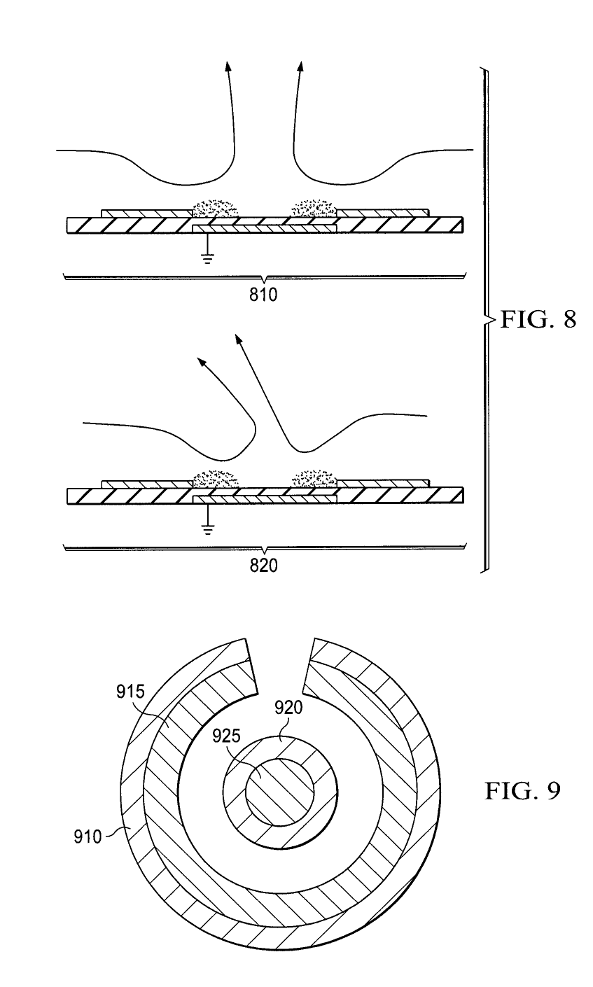

FIG. 8 is a schematic cross-sectional illustration of the embodiment of FIG. 7 that shows that, although the motive force is generally directed orthogonally away from (or toward) the dielectric material, in some configurations and at some points along the embodiment of FIG. 7 that the force may take a path that is non-orthogonal to the dielectric material.

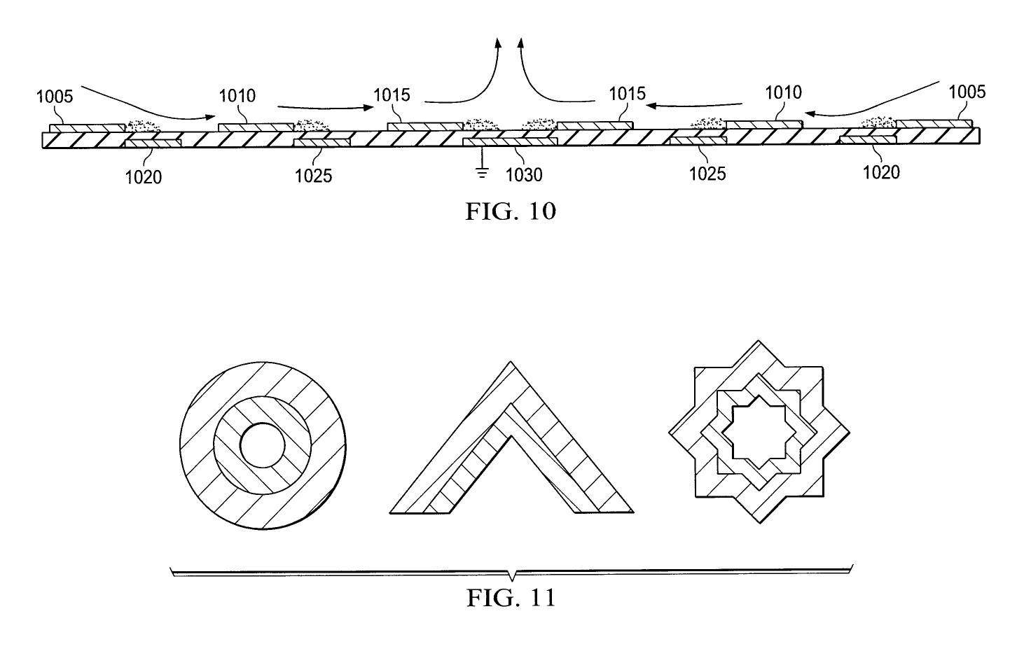

FIGS. 9 and 10 are schematic illustrations of still other arrangements that are generally annular. FIG. 9 contains an illustration of an annular embodiment that includes two upper electrodes 910 and 920 and two lower electrodes 915 and 925. Note that the electrodes 910 and 920 might be electrically isolated from each other or not. The same might also be said with respect to electrodes and 915 and 925.

FIG. 10 contains a cross-sectional view still another annular embodiment, with upper electrodes 1005, 1010, and 1015, and lower electrodes 1020, 1025, and 1030. Note that in some embodiments (e.g., FIGS. 7, 8, and 10) one or more electrodes, e.g., the lower electrode in these figures, is embedded in the dielectric.

FIG. 11 contains some further embodiments, e.g., annular, chevron, and hybrid. Those of ordinary skill in the art will readily be able to devise other shapes and arrangements that generate plasma according to the instant invention.

Note that, although in some embodiments the dielectric is a generally rectangular single planar surface, in other embodiments it might be round, polygonal, etc. Additionally, in still other embodiments the dielectric might be separated into two or more pieces that are interconnected by conductive material. In such an instance, the electrodes of the instant invention might be placed on the same or different pieces of the dielectric. The dielectric and/or associated electrodes might also be non-planar depending on the requirements of a particular application. Thus, for purposes of the instant disclosure it should be understood that the term “dielectric” is applicable to materials that are any shape, that are planar or not, and that might be divided into multiple pieces that are joined by conductive materials.

Further note that for purposes of the instant disclosure, the term “length” should be broadly construed to be any linear dimension of an object. Thus, by way of example, circular dielectrics have an associated length (e.g., a diameter). The width of an object could correspond to a length, as could a diagonal or any other measurement of the dielectric. The shape of the instant electrodes and associated dielectric are arbitrary and might be any suitable shape.

Still further, note that the voltages applied to the top and bottom electrodes will be different. It is important that the voltage differential between the electrodes be sufficient for the generation of plasma, e.g., about 5 to 50 kV as was discussed previously. The positive electrode can either be on the top or the bottom of the dielectric and the orientation might be varied depending on the direction it is desired to have the plasma stream move.

Finally it should be noted that remembered that the term “offset” as used herein should be broadly construed to include cases where there is no overlap between the electrodes (e.g., configurations 425 and 430) as well as cases where there is substantial overlap (e.g., configuration 410). What is important is that the edges of the upper and lower electrodes not be completely coincident, e.g., one electrode or the other should have a free edge (or part of an edge) that does exactly overlay the corresponding electrode on the opposite surface.

Thus, the present invention is well adapted to carry out the objectives and attain the ends and advantages mentioned above as well as those inherent therein. While presently preferred embodiments have been described for purposes of this disclosure, numerous changes and modifications will be apparent to those of ordinary skill in the art. Such changes and modifications are encompassed within the spirit of this invention as defined by the claims.

What is claimed is:

1. A device comprising:
a dielectric layer having opposite sides and a length;
a first and a second electrode each on an opposite side of the dielectric layer and offset along the length of the dielectric layer; and
a voltage source that selectively provides a first voltage on the first electrode and a second voltage on the second electrode such that plasma is generated along the dielectric layer, the plasma providing a decontamination mechanism of adjacent air, and movement of the adjacent air along the dielectric layer.

2. The device of claim 1, further comprising:
a third and a fourth electrode on opposite sides of the dielectric layer and offset along the length of the dielectric layer;
wherein the voltage source selectively provides a third voltage on the third electrode and a fourth voltage on the fourth electrode such that plasma is generated along the dielectric layer, the plasma providing a decontamination mechanism of adjacent air, and movement of the adjacent air along the dielectric layer.

3. The device of claim 2, wherein the first, second, third, and fourth electrodes and the power supply are configured to produce a swirling effect of gasses adjacent the dielectric layer.

4. The device of claim 1, wherein the dielectric layer forms a portion of a decontamination chamber, with the plasma being produced by the electrodes inside the chamber.

5. The device of claim 4, further comprising means for providing a supply of contaminated air into the decontamination chamber.

6. The device of claim 5, further comprising means for evacuating plasma-decontaminated air from the decontamination chamber.

7. A device comprising:
first and second inner electrodes exposed to the inside of a decontamination chamber; and
first and second outer electrodes separated from the first and second inner electrodes by a dielectric material forming a portion of a wall of the decontamination chamber;
wherein the first and second inner electrodes are situated relative to one another and to the respective outer electrodes so as to produce plasma inside the decontamination chamber in response to selective application of sufficient voltage to the first, second, third, and fourth electrodes.

8. The device of claim 7, further comprising an alternating voltage source connected by leads to the first and second inner electrodes and the first and second outer electrodes.

9. The device of claim 7, wherein the first inner and outer electrodes are situated offset relative to one another so as to produce a first motive force of gases within the decontamination chamber when plasma is produced.

10. The device of claim 9, wherein the second inner and outer electrodes are situated offset relative to one another to as to produce a second motive force of gases within the decontamination chamber; and wherein the first and second motive forces of gases are in substantially opposite directions so as to produce a swirling effect of gases within the decontamination chamber.

11. A method comprising:
placing a first electrode on a first side of a dielectric material;
placing a second electrode on a second side of the dielectric material offset from the first electrode;
exposing the first side of the dielectric material to a contaminated gas; and
applying a sufficient voltage differential to the first and second electrodes as to produce a plasma stream on the first side of the dielectric material to decontaminate the gas.

12. The method of claim 11, further comprising providing a decontamination chamber with the first electrode exposed to an interior thereof.

13. The method of claim 12, further comprising introducing the contaminated gas into the decontamination chamber.

14. The method of claim 13, further comprising evacuating decontaminated gas from the decontamination chamber after exposure to plasma.

15. The method of claim 11, further comprising:
placing a third electrode on the first side of a dielectric material;
placing a fourth electrode on the second side of the dielectric material offset from the third electrode;
applying a sufficient voltage differential to the third and fourth electrodes as to produce a second plasma stream on the first side of the dielectric material to decontaminate the gas; and
arranging the first, second, third, and fourth electrodes such that the first and second plasma steams are directed in opposite directions to as to produce a swirling effect of the gas near the dielectric layer.
